(19) United States
(12) Patent Application Publication
(10) Pub. No.: US 2016/0205939 A1
(43) Pub. Date: Jul. 21, 2016

(54) ATRAZINE-DEGRADING BACTERIUM AND METHOD OF UISNG THE SAME FOR SOIL AND PLANT REMEDIATION

(71) Applicant: Biotechnology Center of Shandong Academy of Sciences, Jinan (CN)

(72) Inventors: Dmitry P. Bazhanov, Jinan (CN); Hongmei Li, Jinan (CN); Chengyun Li, Jinan (CN); Jishun Li, Jinan (CN); Hetong Yang, Jinan (CN)

(21) Appl. No.: 14/985,350

(22) Filed: Dec. 30, 2015

(30) Foreign Application Priority Data
Dec. 30, 2014 (CN) 201410839729.8

(51) Int. Cl. A01N 63/00; C12N 1/20

(52) U.S. Cl. CPC A01N 63/00; C12N 1/20

(57) ABSTRACT

The bacterium Arthrobacter ureafaciens liulou 1 (CGMCC 9667) possesses a unique combination of high atrazine-degrading activity, a capability of colonizing plant roots after seed inoculation and traits of a plant growth promoting bacterium. Also disclosed is a method of A. ureafaciens liulou 1 (CGMCC 9667) application for remediation of the polluted soils and plant protection from atrazine.

### CROSS-REFERENCE TO RELATED APPLICATIONS

Pursuant to 35 U.S.C. §119 and the Paris Convention Treaty, this application claims the foreign priority benefit of Chinese Patent Application No. 201410839729.8 filed Dec. 30, 2014, the contents of which, are incorporated herein by reference.

### BACKGROUND OF THE INVENTION

1. Field of the Invention

The present invention relates to an atrazine-degrading bacterial strain that has the ability to colonize plant roots and application of the strain for bioremediation of atrazine-polluted soil.

2. Description of the Related Art

Atrazine (2-chloro-4-(ethylamino)-6-(isopropylamino)-1,3,5-triazine) is the most widely and heavily used herbicide among the broad class of s-triazine compounds. Atrazine and its metabolites desethyl atrazine, desisopropyl atrazine and 2-hydroxyatrazine are commonly detected contaminants of soils, underground and surface streams and basins, where they persist for years and even decades.

Conventionally, the only approach allowing the remediation of contaminated of atrazine soils on large areas was phytoremediation, which includes the growing of plants resistant to the herbicide in the contaminated soil without application of microorganisms. However, this method interferes with harvesting and has a low efficacy of atrazine degradation. In addition, the lack of bacteria capable of rapidly degrading atrazine results in the conversion of atrazine to its dealkylated metabolites, which are persistent in soil and which are toxic to plants.

### SUMMARY OF THE INVENTION

In view of the above-described problems, it is one objective of the invention to provide a novel root-colonizing atrazine-degrading bacterium and applications thereof. The novel atrazine-degrading bacterium can be used for remediation of soil polluted by atrazine or other s-triazine herbicides and for protecting plants from damage caused by application of atrazine or other s-triazine herbicides. The strain possesses a unique combination of properties, such as high atrazine-degrading activity, active motility, an ability to colonize plant roots after seed inoculation, a tolerance to drying and a long-term survival on the inoculated dry seeds, plant growth stimulating activity, an ability to mobilize insoluble phosphates.

The bacterium possesses all of the identifying characteristics of Arthrobacter ureafaciens liulou 1. The strain Arthrobacter ureafaciens liulou 1 has been deposited in China General Microbiological Culture Collection Center (CGMCC) under the No. 9667, on Sep. 16, 2014. Address of CGMCC: No. 1 West Beichen Road, Chaoyang District, Beijing, 100101, China. The strain liulou 1 was identified by 16S rRNA gene phylogenetic analysis and BOX-PCR genotyping. The strain cells appear as Gram-negative rods in young cultures and Gram-positive cocci in old cultures. On an LB agar plate, colonies of the strain liuloul have the following properties: smooth, circular form, convex elevation, pale yellow, opaque appearance. The strain does not produce fluorescein and endospore, and does not hydrolyze starch and liquefy gelatin. The strain is oxidase and catalase positive, and is M.R, V-P negative, esterase (Tween 80), lecithinase, and urease negative. The strain is motile in liquid media, and obviously spread in semisolid agar.

The strain Arthrobacter ureafaciens liulou 1 completely degrades atrazine in a solution with a concentration of 25 mg/L within 1 week without additional aeration.

The strain was isolated from the rhizosphere of maize sampled from the field with a long history of atrazine application. The strain can be found in the bulk and rhizosphere soils, on plant roots. The strain can be isolated by direct spreading of serial dilutions of soil harboring the bacterium on a selective solid medium SM, containing (per liter of distilled water) 0.5 g K₂HPO₄, 0.2 g MgSO₄×7H2O, 0.1 g NaCl, 0.02 g CaCl₂, 2 g D-glucose, 10 mL atrazine stock solution, 5 mL of ZnFe-Cit solution and 13 g of bacteriological agar. The atrazine stock solution contains (per 100 mL of distilled water) 1 mL Tween 80 and 5 g atrazine powder. ZnFe-Cit stock solution contains (per 100 mL of distilled water) 0.04 g ZnSO₄×7H₂O, 0.4 g FeSO₄×7H₂O and 10 g trisodium citrate. A biologically pure culture of the bacterial strain can be obtained by repeated streaking on solid medium SMY, which is medium SM, amended with 0.1 g L−1 yeast extract.

Pure culture of the bacterial strain liulou 1 can be maintained on the solid media SM, SMY (SM amended with 0.1 g/L yeast extract) or other suitable agar media, such as TY (10 g tryptone, 1 g yeast extract, and 0.02 g of CaCl₂ per liter of distilled water) or nutrient agar. As used herein, a “biologically pure culture” means a culture which contains primarily only bacteria from the liulou 1 strain and which is substantially free of other contaminating bacteria.

The present invention also includes a biological agent for remediation of liquids and soils contaminated with atrazine or related s-triazine compounds and for plant protection from atrazine or related s-triazine herbicides. The biological agent can be in the form of a biologically pure culture of Arthrobacter ureafaciens liulou 1 and can include any acceptable liquid or solid carrier that does not interfere with survival of bacteria, such as buffers, water, solutions of various compounds, suspensions, cultures of other microorganisms, powders, soil, peat, etc. The preferred liquid carrier is SM salt buffer containing 0.5 g/L K₂HPO₄, 0.2 g/L MgSO₄.7H₂O, 0.02 g/L CaCl₂ and 0.1 g/L NaCl. The strain Arthrobacter ureafaciens liulou 1 should be present in the biological agent at a sufficient concentration. A sufficient concentration of Arthrobacter ureafaciens liulou 1 is between about 10² and 10¹² CFU/g of the agent. A more preferred sufficient concentration of Arthrobacter ureafaciens liulou 1 is between about 10⁴ and 10⁹ CFU/g of the agent.

The present invention also includes a method of the strain Arthrobacter ureafaciens liulou 1 application for remediation of soils contaminated with atrazine or related s-triazine compounds and for plant protection from atrazine or related s-triazine compounds. The method comprises inoculation of plant seeds with the biological agent and sowing the inoculated seeds to the contaminated soil. After germination of the seeds, the strain A. ureafaciens liulou 1 competitively colonizes seedling roots and produces a population sufficient to degrade soil atrazine, to detoxify the contaminated soil, and to protect plants from the herbicide. The inoculated seeds can be stored prior to sowing, keeping effective amount of the strain liulou 1 for a long period.

The strain Arthrobacter ureafaciens liulou 1 should be present on inoculated seeds in an effective amount. An effective amount of Arthrobacter ureafaciens liulou 1 is between about 10² and 10⁸ CFU/seed. A more preferred effective initial amount of Arthrobacter ureafaciens liulou 1 on seeds to be stored is between about 10⁴ and 10⁸ CFU/seed.

The method of Arthrobacter ureafaciens liulou 1 application through seed inoculation enables a low-cost, not interfering with plant production, area-wide remediation of soils polluted with atrazine and related s-triazine herbicides. Contributing to the reduction of the groundwater and surface reservoirs contamination with atrazine, the method of bioremediation will have high social and ecological benefits.

### BRIEF DESCRIPTION OF THE DRAWINGS

FIG. 1 shows colonies of atrazine-degrading bacteria isolated from maize rhizosphere by direct plating on SM agar. The plate was incubated at 28° C. for 3 days;

FIG. 2 shows a pure culture of the atrazine-degrading strain liulou 1 on SM agar amended with 0.1 g/L yeast extract;

FIG. 3 is a neighbor-joining tree, showing the phylogenetic position of the atrazine-degrading strain liulou 1 among related Arthrobacter species, based on 16S rRNA gene sequences. GenBank accession numbers are shown in parentheses. Bootstrap values (expressed as percentages of 1000 replications) greater than 50% are shown at the branching points. There were a total of 1244 positions in the final dataset. Evolutionary analysis was conducted in MEGA5;

FIG. 4 shows BOX-PCR patterns of the strain lulou 1 (lanes L) and species type strains Arthrobacter ureafaciens CGMCC 1.1897 (lanes Au), Arthrobacter nitroguajacolicus CGMCC 1.7300 (lanes An), and Arthrobacter sulfonivorans CGMCC 1.3977 (lanes As). Lane M contains DL 2,000 DNA Marker (Takara Biotechnology (Dalian) Co., Ltd., China);

FIG. 5 shows ERIC-PCR patterns of the strain lulou 1 (lanes L), and species type strain Arthrobacter ureafaciens CGMCC 1.1897 (lanes Au). Lanes M1 and M2 contain a 100 bp DNA Ladder and a DL 2,000 DNA Marker (Takara Biotechnology (Dalian) Co., Ltd., China) respectively;

FIG. 6 shows the detection of atrazine degradation genes in the strain Arthrobacter ureafaciens liulou 1 bp multiplex PCR:

lane R1, Pseudomonas sp. D3-1l having genes atzA, atzB, and atzC; lane R2, Arthrobacter sp. SD41 having genes atzB, atzC, and trzN; lane L: Arthrobacter ureafaciens liulou 1; and lane M: DL 2,000 DNA Marker;

FIGS. 7A-7D show the colony expansion of Arthrobacter ureafaciens liulou 1 on SA media with different agar concentrations;

FIGS. 8A-8D are diagrams showing the distances and speed of Arthrobacter ureafaciens liulou 1 colony expansion on SA media with different agar concentrations;

FIGS. 9A-9B show colonization of wheat roots by Arthrobacter ureafaciens liulou 1. A clearing zone indicates colonization of the whole root system by atrazine-degrading Arthrobacter ureafaciens liulou 1 after seed inoculation (FIG. 9B). No clearing zone was detected near the roots of control plants from the non-inoculated seeds (FIG. 9A);

FIG. 10 demonstrates that seed inoculation by Arthrobacter ureafaciens liulou 1 protects wheat seedlings from atrazine damage;

FIG. 11 demonstrates that seed inoculation by Arthrobacter ureafaciens liulou 1 protects alfalfa seedlings from atrazine damage; and

FIG. 12 shows a zone of phosphate dissolution (L) produced by Arthrobacter ureafaciens liulou 1 after streaking on an SMP plate.

### DETAILED DESCRIPTION OF THE EMBODIMENTS

For further illustrating the invention, experiments detailing the atrazine-degrading bacterium and applications thereof are described below. It should be noted that the following examples are intended to describe and not to limit the invention.

### Example 1

### Isolation of Strain Liulou 1

The strain liulou 1 was directly isolated on SM agar which contained atrazine as a sole nitrogen source and Tween 80 as a dispersant. The surfactant improved bioavailability of hydrophobic atrazine in SM agar and enabled direct selective isolation of atrazine-degrading bacteria from various sources, including industrial and agricultural soils. Eliminating the bias from routine enrichment procedures, direct plating on SM agar facilitates the isolation of soil- and rhizosphere-competent atrazine degraders.

The strain liulou 1 was isolated from the rhizosphere of maize sampled from the field with a long history of atrazine application at Liulou Village, Binhe Subdistrict, Dingtao County, Heze Prefecture, Shandong Province.

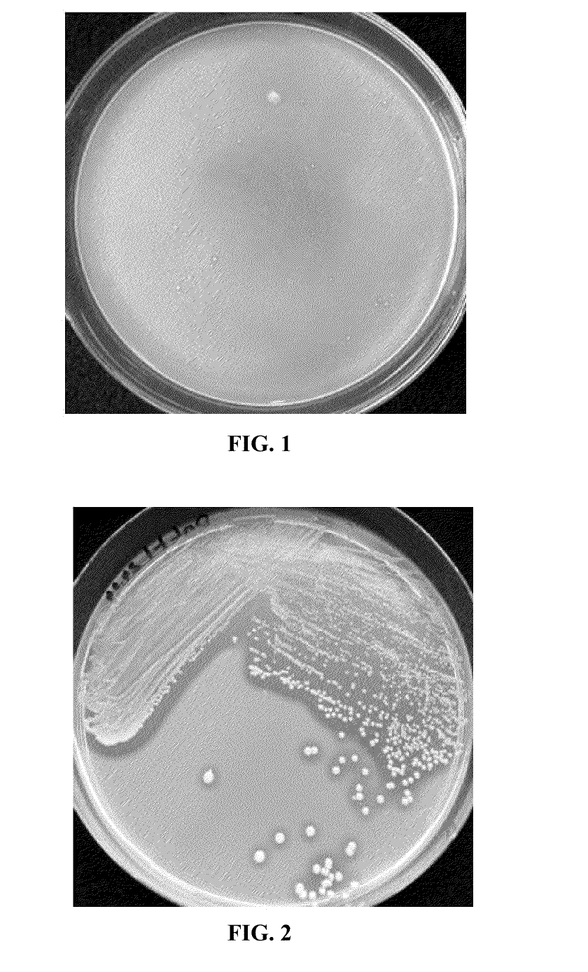

Soil cores 15×10×10 cm (length×width×depth) containing the root system of 1 maize plant were accurately cut out using a surface-sterilized shovel, to avoid disturbing the natural soil structure for the samples. Next day after sampling, the maize roots were carefully retrieved from the cores and the loose soil was removed. The soil associated with root surface was scrapped off and suspended in the buffer (SM medium salt solution). Decimal serial dilutions of the soil suspension were spread on SM agar. Colonies of atrazine degraders were distinguished on SM agar by production of clearing halos due to atrazine degradation after 3 days incubation at 28° C. (as shown in FIG. 1). Colonies from the highest positive dilutions (2nd and 3rd) were repeatedly streaked on SM agar amended with 0.1 g L−1 of yeast extract till pure cultures of atrazine-degrading bacteria were obtained. The strain liulou 1 (shown in FIG. 2) was selected based on the positive results of a complex testing that included: the presence of genes for atrazine degradation, atrazine-degrading capacity, spreading in semisolid agar, colonization of plant roots after seed inoculation, resistance to drying and long-term survival on dry seeds, degradation of atrazine in the soil in association with plants, root elongation activity, capability of mobilizing insoluble phosphates.

### Example 2

### Phylogenetic Identification of Strain Liulou 1

Method

The gene for 16S rRNA were amplified with the primer pair 63KWf (5′CAKGCCTWACACATGCAAGTC3′) and 1387r (5′GGGCGGWGTGTACAAGGC3′). The PCRs were performed in a total volume of 50 μL containing 5 μL of TaKaRa 10× Ex Taq Buffer, (Takara Biotechnology (Dalian) Co., Ltd., China), 2.0 mM MgCl2, 250 μM of each dNTP, 0.5 μM of each primer, 0.5 U of TaKaRa Ex Taq polymerase (Takara Biotechnology (Dalian) Co., Ltd., China) and 1.25 μL of a bacterial lysate as a template. The PCRs were started by denaturation at 95° C. for 3 min. followed by 30 cycles: 94° C. for 1 min., 55° C. for 1 min., 72° C. for 2 min.; followed by extension at 72° C. for 5 min. The amplification products were analyzed by electrophoresis on an agarose gel in 0.5×TBE. The target fragment of about 1.3 kb was cut out from the gel, purified using a TaKaRa MiniBEST Agarose Gel DNA Extraction Kit Ver. 4.0 (Takara Biotechnology (Dalian) Co., Ltd., China) and used as templates in sequencing reactions with the primers 63KWf and 1387r. The automated sequencing was performed on a 3730xl DNA Analyzer (Applied Biosystems, United States). The resulting DNA traces and sequences were checked and corrected manually.

The BLASTn similarity search was performed against GenBank Reference RNA sequences (refsec_rna) database. The phylogenetic analysis was carried out using MEGA5 software package (Tamura et al., 2011). 16S rRNA gene nucleotide sequences of the species type strains from the GenBank database shared more than 98% similarity with the studied one were included into the datasets. Multiple alignments were implemented using CLUSTALW aligner of MEGA5 and then refined by hand. Phylogenies were inferred using Neighbor-Joining algorithm with elimination of all positions containing gaps and missing data.

Results:

Partial (1255 bp) nucleotide sequence of 16S rRNA gene of liulou 1 is shown by the following: SEQ ID NO. 1.

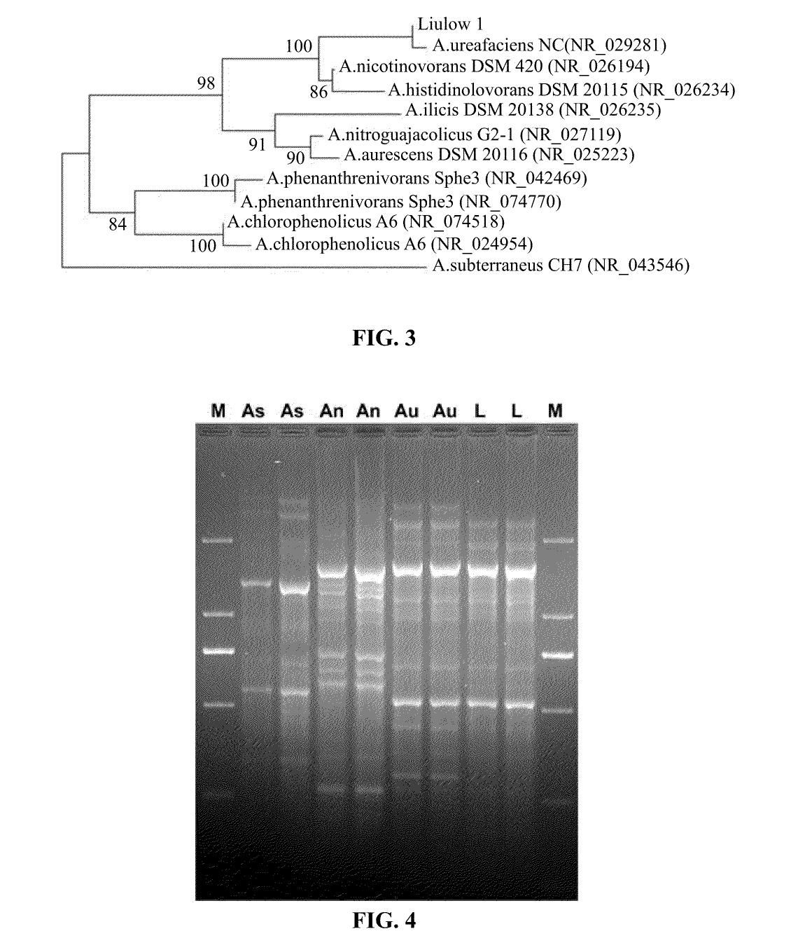

The BLASTn search results gave evidence that strain liulou 1 belonged to the genus Arthrobacter (phylum Actinobacteria, class Actinobacteria, subclass Actinobacteridae, order Actinomycetales, suborder Micrococcineae, family Micrococcaceae). The highest sequence similarity 99.6% was found between strain liulou 1 and the species type strain Arthrobacter ureafaciens NC. The reconstructed phylogenetic tree indicated that strain liulou 1 and the species type strain Arthrobacter ureafaciens NC were closely related (as shown in FIG. 3). The divergence between liulou 1 and Arthrobacter ureafaciens NC did not exceed the divergence between copies of 16S rRNA genes in genomes of the species type strains Arthrobacter phenantrenivorans Sphe3 and Arthrobacter chlorophenolicus A6, supporting affiliation of the strain liulou 1 to Arthrobacter ureafaciens.

### Example 3

### Taxonomic and Individual Identification of Strain Liulou 1 by Repetitive Elements Sequence-Based PCR (Rep-PCR)

Method

Identification of the strain liulou 1 based on Rep-PCR method was conducted by using primers BOXA1R (5′ CTACGGCAAGGCGACGCTGACG 3′) and ERIC2 (5′ AAGTAAGTGACTGGGGTGAGCG 3′). The PCR mixture (20 μL) contained: 2 μL 10× Ex Taq Buffer from Takara Biotechnology (Dalian) Co., Ltd., 2.0 mM MgCl2, 250 μM dNTP, 0.5 μM primers, 0.5 U TaKaRa Ex Taq polymerase, and 0.5 μL of a bacterial lysate as a template. The PCR temperature cycling was as follows: 95° C. predenaturing for 3 min., followed by 4 cycles at 94° C. for 1 min., 40° C. (ERIC-PCR) or 55° C. (BOX-PCR) for 1 min., 68° C. for 8 min.; followed by 30 cycles: 94° C. for 1 min., 52° C. (ERIC-PCR) or 65° C. (BOX-PCR) for 1 min., 72° C. for 2 min. A final extension was performed at 72° C. for 5 min. Products of the amplification were separated by electrophoresis on a 2.0% agarose (Genview, China) gel in 0.5×TBE. The gel was supplemented with 50 μL/L−1 of GoldView Nucleic Acid Stain (Beijing Dingguochangsheng Biotechnology Co., Ltd., China) in order to visualize DNA bands. The Rep-PCR banding patterns were analyzed visually.

Type strains A. ureafaciens CGMCC 1.1897 (=NC), Arthrobacter nitroguajacolicus CGMCC 1.7300, and Arthrobacter sulfonivorans CGMCC 1.3977 were used as reference bacteria.

Results:

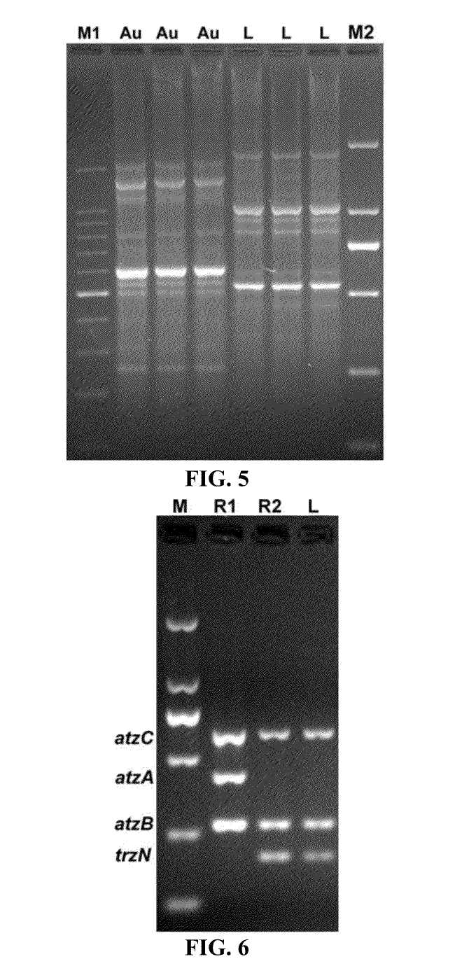

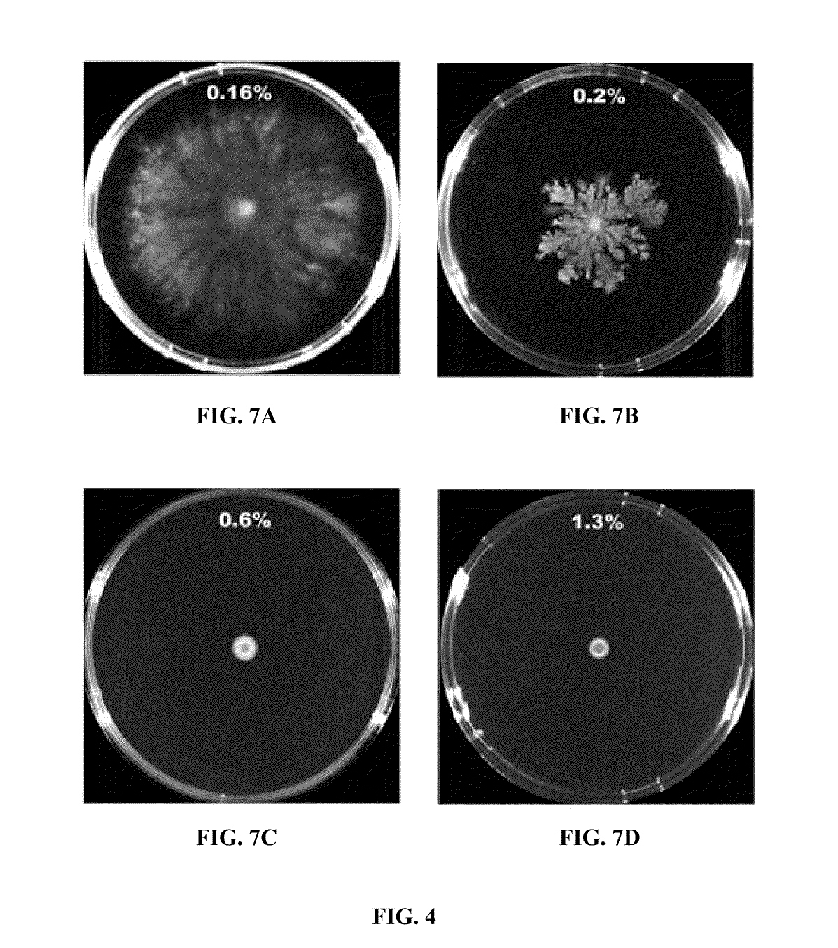

As shown in FIG. 4, BOX-PCR genotyping revealed marked pattern similarity between strain liulou 1 and type strain A. ureafaciens CGMCC 1.1897, indicating their affiliation to the same BOX group. Taking into account that Rep-PCR typing resolves genetic differences between strains within the same species, the strain liulou 1 should be considered a bacterium belonging to the species Arthrobacter ureafaciens.

An ERIC-PCR genotyping detected no similarity between patterns of strain liulou 1 and type strain A. ureafaciens CGMCC 1.1897 (as shown in FIG. 5), indicating that the strains liulou 1 and Arthrobacter ureafaciens CGMCC 1.1897 belong to different ERIC genotypes. The ERIC-PCR genotyping can be used for individual identification of strain liulou 1 and strains having all of the identifying characteristics of the strain liulou 1.

### Example 4

### Identification of the Genes for Atrazine Degradation in Strain Arthrobacter ureafaciens Liulou 1

Method

Identification of genes atzA, atzB, atzC and trzN in strain Arthrobacter ureafaciens liulou 1 was conducted by multiplex PCR using primers pairs atzA250f (TCGCACGGGCGTCAAT)/atzA650r (TGTCACCGCCGTGGTAG), atzB426f (ACCAGTACAACTACAGCCGC)/atzB662r (GGTTTCCGGGTAGGCAATCA), atzCf (GCTCACATGCAGGTACTCCA)/atzCr (TGTACCATATCACCGTTGCCA), and trzN1114f (AATGGCAACCAGGGGATCAG)/trzN1271r (GAGCACCTGACCATTCACGA). The PCR mixture (20 μL) contained: 2 μL 10×Ex Taq Buffer from Takara Biotechnology (Dalian) Co., Ltd., 2.0 mM MgCl2, 250 μM dNTP, 0.2 μM primers, 0.5 U TaKaRa Ex Taq polymerase, and 0.5 μL bacterial lysate as a template. The PCR temperature program was as follows: denaturation at 95° C. for 3 min; then 25 cycles consisted of 94° C. for 1 min, 62° for 30 sec., 72° C. for 1 min; and final extension at 72° C. for 2 min. The strains Pseudomonas sp. D3-1l, bearing the genes atzA, -B and -C, and Arthrobacter sp. SD41 harboring atzB, -C and trzN were used as reference bacteria.

Results:

As shown in FIG. 6, the multiplex PCR with DNA of the strain liulou 1 as a template resulted in amplification of atzB, -C and trzN gene fragments with the predicted sizes of 275, 626, and 196 base pairs respectively. The reaction for atzA was negative. The results gave evidence that strain A. ureafaciens liulou 1 harbors the atrazine degradation genes trzN, atzB, and atzC. The presence of trzN gene suggests that A. ureafaciens liulou 1 produces TrzN chlorohydrolase that capable of dechlorinating wide range of s-triazine compounds (including desethyl atrazine, desisopropyl atrazine, simazine, cyanazine, propazine, terbuthylazine, prometryn, ametryn, terbutryn, simetone, etc.). The hydrolases AtzB and AtzC convert the hydroxy derivatives to non-toxic cyanuric acid, readily mineralized by large number of microorganisms under a wide variety of natural conditions. This example demonstrates the presence of atrazine degradation genes trzN, atzB, and atzC coding enzymes for the degradation of atrazine and other s-triazine compounds.

### Example 5

### Degradation of Atrazine by Arthrobacter ureafaciens Liulou 1

Method: Twenty five mL of liquid medium SM25 (SM with concentration of atrazine reduced to 25 mg L−1) in 250 mL Erlenmeyer flask were inoculated by suspending one 3 days old colony of the strain Arthrobacter ureafaciens liulou 1 growing on SM agar amended with 0.1 g L−1 of yeast extract. The inoculated and control (sterile medium SM25) flasks were incubated without shaking at 28° C. for 1 week. After incubation, the culture was centrifuged and the clear culture liquid was transferred to 50 mL polypropylene tube. The culture liquid and the incubated sterile medium were kept at minus 20° C. until analysis. The second control was sterile medium SM25 kept at −20° C. from the beginning of the experiment.

The liquids were analyzed by high performance liquid chromatography-tandem mass spectrometry (HPLC-MS/MS) on an UltiMate 3000 HPLC system in combination with TSQ Vantage Triple Quadrupole Mass Spectrometer (Thermo Fisher Scientific, USA). Atrazine and metabolites were separated on a column Syncronis HILIC (250 mm×4.6 mm, particle size 5 μm). A binary mobile phase gradient (20 mM ammonium formate and CH3CN) was used for the pesticide separation. Mass transitions 216.1→69.0, 216.1→104.0 and 216.1→174.0 were used for monitoring atrazine.

Results:

HPLC-MS/MS analysis of the stored sterile medium SM25 and incubated sterile medium SM25 revealed atrazine concentration 25 mg L−1 and 26 mg L−1 respectively. Slight increase in the concentration of atrazine in the incubated medium seemed to be caused by water evaporation from the medium during 1 week incubation. No atrazine was detected in the liquid inoculated with liulou 1 (the detection limit was 10 ng L−1), indicating complete degradation of atrazine and a high atrazine-degrading activity of the bacterium Arthrobacter ureafaciens liulou 1.

### Example 6

### Examination of Motility in Arthrobacter ureafaciens Liulou 1

Method

Motility of Arthrobacter ureafaciens liulou 1 was examined on the surfaces of agarized medium SA (0.5 g/L K₂HPO₄, 0.2 g/L MgSO₄ 7H₂O, 0.02 g/L CaCl₂, 0.1 g/L NaCl and 0.1 g/L L-asparagine) with agar concentrations of 1.3%, 0.6%, 0.2%, and 0.16%. A single colony of Arthrobacter ureafaciens liulou 1 was suspended in 50 μL SM buffer (0.5 g/L K₂HPO₄, 0.2 g/L MgSO₄.7H₂O, 0.02 g/L CaCl₂, and 0.1 g/L NaCl) to produce a suspension with the density of nearly 5×10⁹ CFY/mL. 2 μL aliquots of the bacterial suspension were dropped onto centers of SA medium plates with different agar concentrations. The plates were incubated at 28° C. for 7 days. Colony expansion was daily measured during the incubation period.

Results:

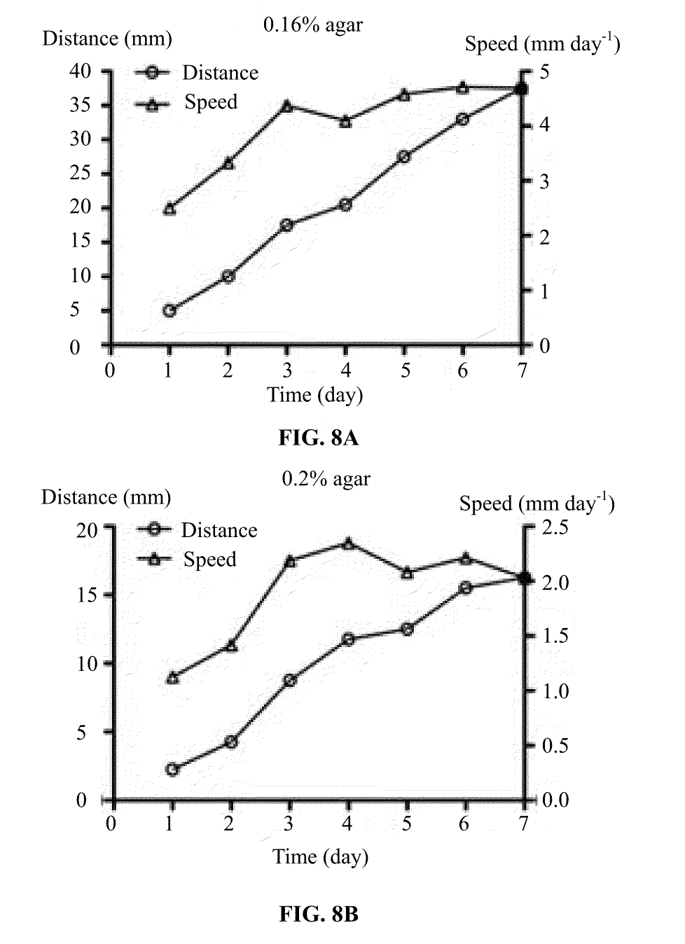

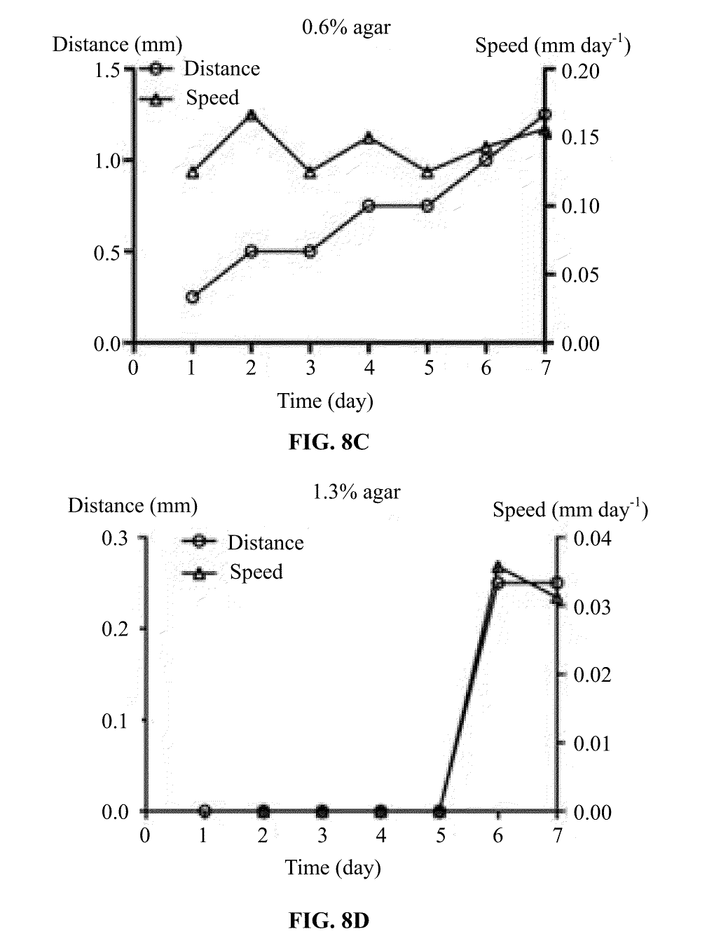

As shown in FIGS. 7A-7D, colonies of Arthrobacter ureafaciens liulou 1 obviously expanded in semisolid (0.16% and 0.2% agar) SA medium. As shown in FIGS. 8A-8D, the highest expansion speed was observed at agar concentration of 0.16%, indicating the swimming motility. A slow colony expansion was detected on the surfaces of SA medium with agar concentrations of 0.6% and 1.3%. The observed colony morphology and slow expansion rate (as shown in FIGS. 7A-7D and 8A-8D) are consistent with swarming motility. This example demonstrates the motility ability in Arthrobacter ureafaciens liulou 1 that is required for active colonization of plant root after seed inoculation.

### Example 7

### Colonization of Plant Roots by Arthrobacter ureafaciens Liulou 1

Method

The capacity of A. ureafaciens liulou 1 to colonize plant roots was assessed in soil-sand assay. Glass tubes (25×200 mm) were filled with previously passed through a 2 mm sieve sand to a depth of 6.5 cm. 7 mL of atrazine solution in distilled water of a determined concentration (as shown in Table 1) were added to each tube. The sand was overlaid with 2 mL field soil harboring no atrazine degrading bacteria, as it was previously determined by using the enrichment procedure and PCR assay for detection of atrazine degradation genes in soil DNA.

Germinated plant seeds with radicles <2 mm were inoculated by soaking in a suspension of A. ureafaciens liulou 1 in the dilution buffer for 1 h. Various densities of seed inoculation were produced by varying the suspension density. One inoculated seed of maize or wheat, or 3 alfalfa seeds were added per tube. Non-inoculated seeds were placed into control tubes. The seeds in tubes were covered with 2 mL soil. The tubes were capped with foil caps, placed into racks and incubated under sunlight at ambient temperature (22-26° C.) without adding water. The rack sides were closed with foil to prevent overheating of the tubes.

To determine the inoculation density, bacteria were washed from the seeds. 10 wheat seeds or 20 alfalfa seeds were placed in a 2 mL polypropylene tube with 1.0 mL buffer. Maize seeds were washed in 5 mL polypropylene tubes (5 seeds with 2.0 mL buffer). The tubes with seeds were secured in a MO BIO Vortex Adapter assembled on Vortex-Genie® 2 Vortex (MO BIO Laboratories, Inc., USA) and vortexed at maximal speed for 10 min. The resulting suspension was considered a “0” dilution. Serial decimal dilutions were prepared and spread on agar SM. The colonies with clearing halos were counted after 4 days' incubation at 28° C.

To evaluate densities of root-associated populations, the root systems were harvested 23 days post seeding. The tubes were accurately broken and the roots were cut under the soil. The root segments were placed in Petri dishes with 30 mL buffer for 20 minutes to detach adhered sand particles. Then, the root segments were placed on filter paper for 5 sec. and transferred to a 2 mL polypropylene tube with 1.0 mL buffer for wheat and alfalfa roots, or to a 5 mL polypropylene tube with 2.0 mL buffer for maize roots. The tubes with root sections were secured in a MO BIO Vortex Adapter assembled on Vortex-Genie® 2 Vortex (MO BIO Laboratories, Inc., USA) and vortexed at maximal speed for 10 min. The resulting suspensions were considered a “0” dilutions. Serial decimal dilutions were prepared and spread on SM agar. The colonies with clearing halos were counted after 4 days' incubation at 28° C. After washing, the root segments were blotted on filter paper and weight immediately. The densities of Arthrobacter ureafaciens liulou 1 root-associated populations were calculated per g of fresh roots in 5 replicates per treatment.

To demonstrate the presence and distribution of A. ureafaciens liulou 1 on roots, the washed and blotted root systems of wheat were placed on SM agar and incubated for 3 days at 28° C.

Results:

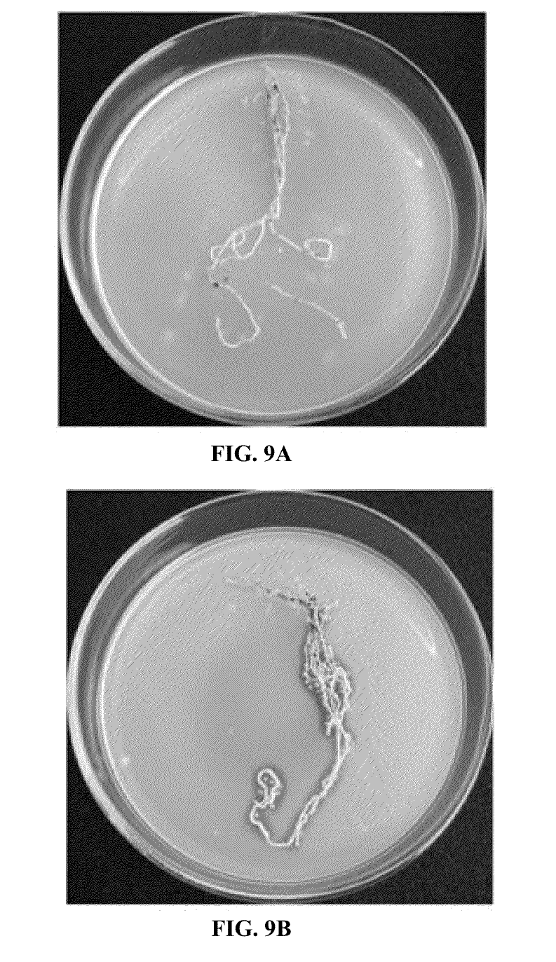

The test results (as shown in Table 1) demonstrated that, after seed inoculation, the strain Arthrobacter ureafaciens liulou 1 was recovered from roots of all the plants tested. Densities of Arthrobacter ureafaciens liulou 1 populations associated with roots of maize and wheat were around 10⁶ CFU/g fresh root. Within the range from 10⁷ to 10³ CFU per seed, the inoculum size did not affect the population density of Arthrobacter ureafaciens liulou 1 on wheat roots. Clearing halos produced near the wheat roots incubated on SM agar indicated that Arthrobacter ureafaciens liulou 1 colonized the whole root system after seed inoculation (as shown in FIGS. 9A-9B).

After inoculation of alfalfa seeds with the density of 10⁵ CFU per seed, the root-associated population of Arthrobacter ureafaciens liulou 1 was about 10⁵ CFU/g fresh root. The inoculation with significantly (1,000-fold) reduced density resulted in only slightly (less than 10-fold) reduced colonization of alfalfa roots by A. ureafaciens liulou 1. This example demonstrates the colonization of plant roots by Arthrobacter ureafaciens liulou 1 and growth of its root-associated population after seed inoculation.

TABLE 1
Colonization of plant roots by Arthrobacter ureafaciens
liulou 1 after Seed Inoculation

| | | | | Min-max root |
|---|---|---|---|---|
| | Atrazine | Total | | populations |
| | concen- | amount of | Inoculation | of Arthrobacter |
| | tration | atrazine | density | ureafaciens liulou 1 |
| Plant | (μg/mL) | (μg/tube) | (CFU/seed) | (CFU/g fresh root) |
| Corn | 25.0 | 175.0 | Non-inoculated | <10² |
| | | | (7.5 ± 0.3) × 10⁷ | 4.7 × 10⁵-1.2 × 10⁶ |
| Wheat | 25.0 | 175.0 | Non-inoculated | <10² |
| | | | (3.2 ± 0.1) × 10⁷ | 0.7 × 10⁶-1.9 × 10⁶ |
| | | | (4.7 ± 0.2) × 10⁶ | 0.7 × 10⁶-3.9 × 10⁶ |
| | | | (6.2 ± 0.3) × 10³ | 0.9 × 10⁶-2.4 × 10⁶ |
| Alfalfa | 2.5 | 17.5 | Non-inoculated | <10² |
| | | | (5.3 ± 0.2) × 10⁵ | 1.8 × 10⁵-2.3 × 10⁵ |
| | | | (1.2 ± 0.1) × 10² | 2.3 × 10⁴-6.5 × 10⁴ |

### Example 8

### Survival of Arthrobacter ureafaciens Liulou 1 on Dry Seed

Method

Dry seeds of wheat and alfalfa were inoculated by soaking in a suspension of Arthrobacter ureafaciens liulou 1 for 1 h. Then, excess moisture was removed by blotting on filter paper, and the seeds were air dried at room temperature (23-26° C.) for 1 day. The seeds were kept in Petri dishes in a dark place at room temperature for more than 3 months. The CFU density of Arthrobacter ureafaciens liulou 1 on seeds was regularly evaluated in the manner described in Example 7.

Results:

Amounts of bacteria survived on the dry seed are presented in Table 2. It was found that 1 day air-drying reduced seed population of Arthrobacter ureafaciens liulou 1 nearly 10-fold. However, the recoverable population had significantly grown after 1 week storage of the inoculated seeds. This indicated that the observed population decline was from a desiccation stress reducing the recoverability of liulou 1 rather than from the death of the inoculant cells. The following evaluations demonstrated that the density of A. ureafaciens liulou 1 on seeds dropped gradually during storage period. Seed populations of liulou 1 after more than 3 months storage were still higher than the effective amount required for competitive root colonization and atrazine degradation in soil (as determined in Examples 7, 9 and 10). This example demonstrates the tolerance of Arthrobacter ureafaciens liulou 1 seed population to drying that enables storage of the inoculated seeds for at least 3 months.

TABLE 2
Dynamics of Arthrobacter ureafaciens
liulou 1 Populations on Dry Seed

| | | Seed populations of liulou 1 | |
|---|---|---|---|
| | | (CFU/seed) | |
| Storage time after inoculation | | Wheat | Alfalfa |
| 0 | (wet seed after inoculation) | (8.0 ± 0.3) × 10⁵ | (3.8 ± 0.2) × 10⁵ |
| 1 | day | (6.3 ± 0.4) × 10⁴ | (5.3 ± 0.4) × 10⁴ |
| 2 | days | (2.3 ± 0.1) × 10⁵ | Not analyzed |
| 1 | week | (3.9 ± 0.1) × 10⁵ | (2.3 ± 0.2) × 10⁵ |
| 2 | weeks | (3.6 ± 0.2) × 10⁵ | (1.4 ± 0.1) × 10⁵ |
| 5 | weeks | (2.0 ± 0.1) × 10⁵ | (1.0 ± 0.1) × 10⁵ |
| 7 | weeks | (1.1 ± 0.1) × 10⁵ | (7.0 ± 0.5) × 10⁴ |
| 9 | weeks | (6.4 ± 0.2) × 10⁴ | (3.1 ± 0.2) × 10⁴ |
| 11 | weeks | (3.1 ± 0.1) × 10⁴ | (1.4 ± 0.1) × 10⁴ |
| 15 | weeks | (1.0 ± 0.1) × 10⁴ | (7.5 ± 0.4) × 10³ |

### Example 9

### Colonization of Wheat Roots by Arthrobacter ureafaciens Liulou 1 after Drying and Storage of the Inoculated Seeds

Method

The capacity of Arthrobacter ureafaciens liulou 1 to colonize wheat roots after drying and storage of the inoculated seeds was assessed in a pot experiment. Seeds of wheat were inoculated by soaking in a suspension of Arthrobacter ureafaciens liulou 1 for 1 h, air-dried and stored at room temperature (23-26° C.) for 2 month. The seed population of Arthrobacter ureafaciens liulou 1 dropped to (6.4±0.2)×10⁴ CFU/seed during the storage period. The 9 cm diameter pots contained field soil of 13% moisture (300 g dry weight equivalents) harboring no atrazine-degrading bacteria. 70 mL distilled water with 1.75 mg of dissolved atrazine were added to half of the pots, the other half (control pots) received water without atrazine. The inoculated and non-inoculated seeds were sown to the atrazine-treated and non-treated soils. The wheat plants were grown under sunlight with natural photoperiod of Jinan in September to October, 20-26° C. day temperatures and 15-20° C. at night. Wheat roots growing insides soil cores were harvested on 28th day post seeding. The root segments were processed in the manner described in the Example 7. The densities of Arthrobacter ureafaciens liulou 1 root-associated populations were calculated per g of fresh roots in 4 replicate pots per treatment.

Results:

The test result (as shown by Table 3) demonstrated that drying and storage of the inoculated seeds did not affect the capacity of Arthrobacter ureafaciens liulou 1 to colonize wheat roots. The density of Arthrobacter ureafaciens liulou 1 root populations in soil treated with atrazine was higher than that in soil without atrazine. This example demonstrates that Arthrobacter ureafaciens liulou 1 colonizes plant roots after seed inoculation followed by drying and storage of the inoculated seeds.

TABLE 3
Colonization of Wheat Roots by Arthrobacter ureafaciens
liulou 1 after Seed Inoculation

| | | Min-max root populations of |
|---|---|---|
| Atrazine dose | Inoculum density | Arthrobacter ureafaciens liulou 1 |
| (mg/pot) | (CFU/seed) | (CFU/g of fresh roots) |
| None | Non-inoculated | <10² |
| | (6.4 ± 0.2) × 10⁴ | 1.0 × 10⁴-4.9 × 10⁵ |
| 1.75 | Non-inoculated | <10² |
| | (6.4 ± 0.2) × 10⁴ | 8.1 × 10⁵-1.8 × 10⁶ |

### Example 10

### Remediation Test

Method

The atrazine-degrading capacity of Arthrobacter ureafaciens liulou 1 was assessed in the soil-sand assay described in the Example 7. The tubes were incubated for 30 days and then kept at −20° C. prior analysis. The control tubes were placed into the freezer at the start of the experiment and kept at −20° C. prior analysis.

Atrazine content was determined in 4 replicate tubes per treatment. Atrazine was extracted with 30 mL of 80:20 (v/v) methanol/25 mM ammonium acetate adjusted to pH 8.0 from the whole volume of the soil-sand sample in a tube. The suspension was sonicated for 50 min in an ultrasonic bath and centrifuged at 8000 g for 15 min., and the supernatant was transferred to a 50-mL polypropylene tube. The extraction procedure was repeated, and the supernatants were combined. The supernatant was evaporated to <5 mL at 50° C. Atrazine was selectively extracted by solid phase extraction on a C18 column, preconditioned with 3 mL each of ethyl acetate, methanol and purified water. The column was dried under negative pressure for 90 min, and then atrazine was eluted with 5 mL ethyl acetate. The extract was concentrated to a final volume of 10 μL by gentle nitrogen stream evaporation. Volume of the extract was adjusted to 1 mL with methanol. The sample was analyzed by high performance liquid chromatography-tandem mass spectrometry (HPLC-MS/MS) as it has been described in Example 5.

Results:

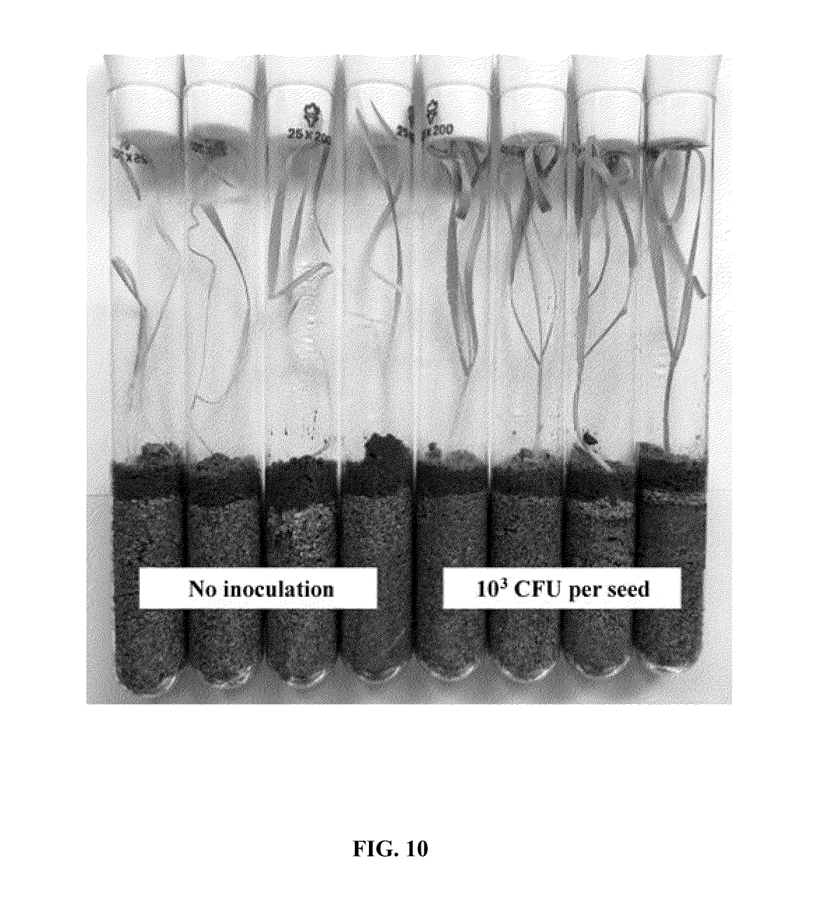

Atrazine injury symptoms became apparent on non-inoculated wheat seedlings during the last week of incubation. Severe leaf chlorosis and necrosis resulted in death of all the control seedlings by the end of incubation. No herbicide injury occurred in the plants from seeds inoculated by Arthrobacter ureafaciens liulou 1, both at the high and low densities of the inoculant (as shown by FIG. 10).

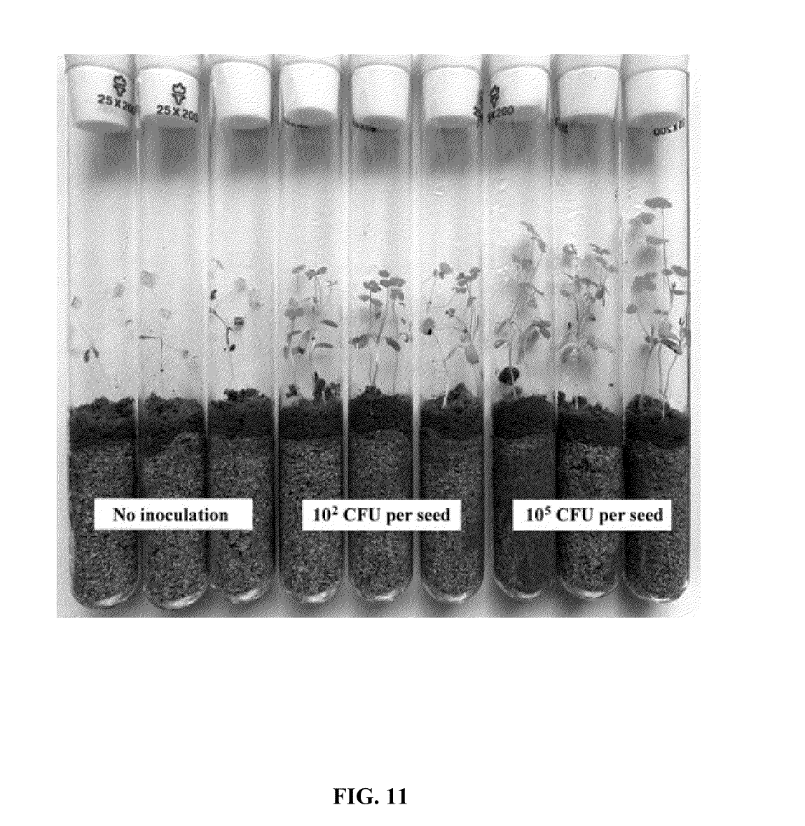

Injury symptoms from atrazine appeared on small alfalfa plants from 15 to 17 days post seeding and resulted in seedling death during the following week. Alfalfa plants treated with A. ureafaciens liulou 1 developed no marked chlorosis till the end of the incubation (30 days) regardless the density of seed inoculation. However, seeds inoculated with 10⁵ CFU per seed gave more vigorous plants than those inoculated with 10² CFU (as shown by FIG. 11).

HPLC-MS/MS analysis of the soil-sand extracts gave evidence that Arthrobacter ureafaciens liulou 1 in association with wheat roots degraded 99.7-99.8% of the applied atrazine during 1 month after sowing of the inoculated seeds (as shown in Table 4). The association of Arthrobacter ureafaciens liulou 1 with alfalfa degraded 70.2-75.8% of atrazine applied. Atrazine degradation in the tubes with non-inoculated wheat and alfalfa plants was 25.7% and 36.3% respectively. This example demonstrates that seed inoculation with A. ureafaciens liulou 1 protects exposed to atrazine plants from the herbicide and results in the degradation of atrazine during plant growth.

TABLE 4
Degradation of Atrazine by Arthrobacter ureafaciens
liulou 1 in Association with Wheat or Alfalfa

| | | | | Atrazine | |
|---|---|---|---|---|---|
| Atrazine dose | Test | | Inoculation density | recovered | Atrazine |
| (μg/tube) | tube | Plant | (CFU/seed) | (μg/tube) | degradation |
| 175.0 | Control | None | Non-inoculated | 145.3 ± 11.2 | 0% |
| | W* | | | | |
| | W1 | | Non-inoculated | 108.0 ± 11.4 | 25.7% |
| | W2 | Wheat | (4.7 ± 0.2) × 10⁶ | 0.37 ± 0.14 | 99.7% |
| | W3 | | (6.2 ± 0.3) × 10³ | 0.25 ± 0.06 | 99.8% |
| 17.5 | Control | None | Non-inoculated | 12.4 ± 1.6 | 0% |
| | A* | | | | |
| | A1 | | Non-inoculated | 7.9 ± 2.3 | 36.3% |
| | A2 | Alfalfa | (5.3 ± 0.2) × 10⁵ | 3.0 ± 0.9 | 75.8% |
| | A3 | | (1.2 ± 0.1) × 10² | 3.7 ± 1.1 | 70.2% |

*The control tubes W and A were kept at −20° C. from the beginning of the experiments.

### Example 11

### Promotion of Root Growth by Arthrobacter ureafaciens Liulou 1

Method

Filter paper (15×60 cm, 2 layers) and wax paper (10×70 cm) were prepared.

Seeds of alfalfa were inoculated with Arthrobacter ureafaciens liulou 1, dried and stored in the manner described in Example 8. A sheet of filter paper (15×60 cm, 2 layers) was placed on a desk and moistened with 30 mL distilled water. The inoculated or non inoculated (control) dry seeds of alfalfa were placed on the wet filter paper (1.0 cm from the sheet edge with 2 cm intervals, 30 seeds per sheet) and covered with a strip of wax paper (10×70 cm). The paper “sandwiches” were carefully rolled and placed (seeds up) into 500 mL plastic cylinders with 33 mL of distilled water. The cylinders were sealed with parafilm and incubated in the dark at room temperature (26-27° C.). The root length was measured after 5-day incubation.

Results:

As shown in Table 5, the root length of seedlings from dry inoculated seeds of alfalfa was significantly higher than that of the control seedlings. The root length of seedlings from the inoculated seeds stored for 7, 9, and 11 weeks was increased by 136.1%, 33.1%, and 50% respectively compared to the control groups. This example demonstrates that inoculation of alfalfa seed with Arthrobacter ureafaciens liulou 1 significantly promotes radical growth.

TABLE 5
Growth Promotion of Alfalfa Radicles by Inoculation of
Dry Seeds with Arthrobacter ureafaciens liulou 1
Root length (mm)

| | | | | Percent |
|---|---|---|---|---|
| | | Seedlings | | increase in |
| Storage | Inoculation | from the | | root length |
| period | density | inoculated | Control | due to the |
| (weeks) | (CFU/seed) | seeds | seedlings | inoculation |
| 7 | (7.0 ± 0.5) × 10⁴ | 49.1 ± 6.6 | 20.8 ± 5.5 | 136.1 |
| 9 | (3.1 ± 0.2) × 10⁴ | 43.1 ± 4.2 | 32.6 ± 5.0 | 33.1 |
| 11 | (1.4 ± 0.1) × 10⁴ | 48.9 ± 5.1 | 32.6 ± 4.8 | 50.0 |

### Example 12

### Phosphate Solubilization Test

Methods

Phosphate solubilization test was conducted on a SMP solid medium plate. The SMP medium was SM agar without atrazine stock solution, and amended with 0.5 g/L NH4Cl and 0.1 g/L yeast extract. CaHPO4 was added to SMP agar as 10% powder suspension in 1% Tween 80 to a final concentration of 5 g/L. An agar concentration in SMP solid medium was 17 g/L. To detect phosphate solubilization, atrazine-degrading isolates were streaked on the SMP agar sectors, 9 sectors per plate. The plates were incubated at 28° C. for 1 week. After incubation, the bacteria were removed from the agar surface to observe zones of CaHPO4 solubilization.

Results:

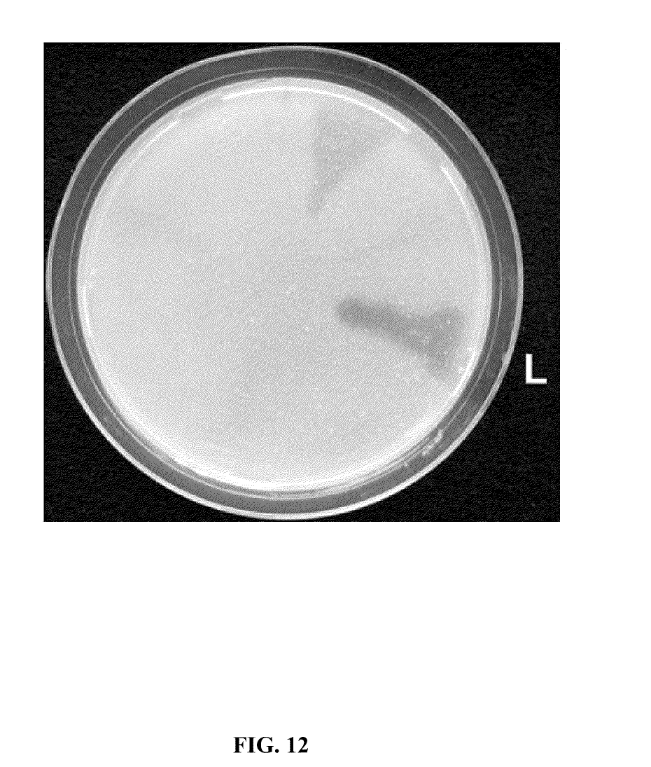

As shown in FIG. 12, Arthrobacter ureafaciens liulou 1 dissolved CaHPO4 after 1 week cultivation on SMP agar, demonstrating the phosphate-mobilizing activity.

Sequence Listing:

Partial (1255 bp) nucleotide sequence of 16S rRNA gene of liulou 1 is shown by the following: SEQ ID NO. 1:

| | |
|---|---|
| 1 | TAGTGGCGAA CGGGTGAGTA ACACGTGAGT AACCTGCCCT TGACTCTGGG ATAAGCCTGG |
| 61 | GAAACTGGGT CTAATACCGG ATATGACTCC TCATCGCATG GTGGGGGGTG GAAAGCTTTT |
| 121 | TGTGGTTTTG GATGGACTCG CGGCCTATCA GCTTGTTGGT GGGGTAATGG CCTACCAAGG |
| 181 | CGACGACGGG TAGCCGGCCT GAGAGGGTGA CCGGCCACAC TGGGACTGAG ACACGGCCCA |
| 241 | GACTCCTACG GGAGGCAGCA GTGGGGAATA TTGCACAATG GGCGAAAGCC TGATGCAGCG |
| 301 | ACGCCGCGTG AGGGATGACG GCCTTCGGGT TGTAAACCTC TTTCAGTAGG GAAGAAGCCC |
| 361 | TCTTTGGGGG TGACGGTACT TGCAGAAGAA GCGCCGGCTA ACTACGTGCC AGCAGCCGCG |
| 421 | GTAATACGTA GGGCGCAAGC GTTATCCGGA ATTATTGGGC GTAAAGAGCT CGTAGGCGGT |
| 481 | TTGTCGCGTC TGCTGTGAAA GACCGGGGCT CAACTCCGGT TCTGCAGTGG GTACGGGCAG |
| 541 | ACTAGAGTGC AGTAGGGGAG ACTGGAATTC CTGGTGTAGC GGTGAAATGC GCAGATATCA |
| 601 | GGAGGAACAC CGATGGCGAA GGCAGGTCTC TGGGCTGTAA CTGACGCTGA GGAGCGAAAG |
| 661 | CATGGGGAGC GAACAGGATT AGATACCCTG GTAGTCCATG CCGTAAACGT TGGGCACTAG |
| 721 | GTGTGGGGGA CATTCCACGT TTTCCGCGCC GTAGCTAACG CATTAAGTGC CCCGCCTGGG |
| 781 | GAGTACGGCC GCAAGGCTAA AACTCAAAGG AATTGACGGG GGCCCGCACA AGCGGCGGAG |
| 841 | CATGCGGATT AATTCGATGC AACGCGAAGA ACCTTACCAA GGCTTGACAT GGACCGGAAA |
| 901 | GACCTGGAAA CAGGTGCCCC GCTTGCGGCC GGTTTACAGG TGGTGCATGG TTGTCGTCAG |
| 961 | CTCGTGTCGT GAGATGTTGG GTTAAGTCCC GCAACGAGCG CAACCCTCGT TCTATGTTGC |
| 1021 | CAGCGGTTCG GCCGGGGACT CATAGGAGAC TGCCGGGGTC AACTCGGAGG AAGGTGGGGA |
| 1081 | CGACGTCAAA TCATCATGCC CCTTATGTCT TGGGCTTCAC GCATGCTACA ATGGCCGGTA |
| 1141 | CAAAGGGTTG CGATACTGTG AGGTGGAGCT AATCCCAAAA AGCCGGTCTC AGTTCGGATT |
| 1201 | GGGGTCTGCA ACTCGACCCC ATGAAGTCGG AGTCGCTAGT AATCGCAGAT CAGCA |

### Example 13

### Degradation of Atrazine, Desethyl Atrazine and Desisopropyl Atrazine by Arthrobacter ureafaciens Liulou 1

Method

To evaluate degradation of atrazine and its derivatives by Arthrobacter ureafaciens liulou 1, 25 mL of liquid medium M3 (the medium SM containing atrazine, desethyl atrazine and desisopropyl atrazine, 5 mg/L of each compound) in 250 mL Erlenmeyer flasks were inoculated with Arthrobacter ureafaciens liulou 1 to a density of 10⁴ CFU/mL. The inoculated and control (sterile medium M3) flasks were incubated without shaking at 28° C. for 1 week. After incubation, the culture was centrifuged and the clear culture liquid was transferred to a 50 mL polypropylene tube. Samples of the liquid and the incubated sterile medium were kept at −20° C. until analysis.

The liquids were analyzed by high performance liquid chromatography-tandem mass spectrometry (HPLC-MS/MS) on an UltiMate 3000 HPLC system interfaced to a TSQ Vantage Triple Quadrupole Mass Spectrometer (Thermo Fisher Scientific, USA). Atrazine and metabolites were separated on a Thermo Scientific™ Acclaim™ 120 C18 column (250 mm×4.6 mm, particle size 5 μm) with a flow rate of 0.6 mL/min. Methanol (A) and 0.1% aqueous formic acid solution (B) were used for HPLC gradient elution. The program of gradient elution was 0 to 6 min, 5% A; 20 to 25 min, 90% A; 25.1 to 30 min, 5% A. MS/MS analysis was performed in positive electrospray ionization mode (ESI+) and transitions were measured in multiple reaction monitoring (MRM). Parameters of MS/MS and limits of quantification for the analytes are specified in Table 6.

TABLE 6
MS/MS parameters and limits of quantification (LOQ)

| | Retention | | Product ions | | | Analytical | LOQ in |
|---|---|---|---|---|---|---|---|
| | time | Precursor | [confirmation | Collision | S-lens | LOQ | soil |
| Analyte | (min) | ion (m/z) | ions] (m/z) | energies (eV) | offset | (ng/mL) | (ng/g)* |
| Atrazine | 24.16 | 216 | 174, [104, 68] | 18, [29, 34] | 100 | 0.01 | 0.045 |
| Atrazine-2- | | | | | | | |
| hydroxyl | 18.02 | 198 | 156, [86, 69] | 17, [23, 33] | 92 | 0.01 | 0.045 |
| Atrazine- | | | | | | | |
| desethyl | 21.24 | 188 | 146, [104, 68] | 17, [25, 30] | 81 | 0.1 | 0.45 |
| Atrazine- | | | | | | | |
| desisopropyl | 19.46 | 174 | 132, [104, 68] | 16, [22, 29] | 96 | 0.5 | 2.25 |
| Atrazine- | | | | | | | |
| desethyl- | | | | | | | |
| desisopropyl | 14.27 | 146 | 110, [104, 68] | 16, [18, 27] | 59 | 0.2 | 0.90 |

*Indicated LOQ in soil could be improved by concentration of the extracts.

Results

HPLC-MS/MS analysis of the cultural liquids detected trace amounts of atrazine (<0.01 ng/mL), desethyl atrazine (<0.1 ng/mL) and desisopropyl atrazine (<0.5 ng/mL), indicating >99.99% degradation of the analytes. No significant degradation of the compounds was detected in the non-inoculated control liquid. This example demonstrates nearly complete degradation of atrazine, desethyl atrazine and desisopropyl atrazine in a solution at their initial concentration as low as 5 mg/L that is close to the ecologically relevant concentration.

### Example 14

### Remediation of Atrazine-Contaminated Soil by Using Seed Inoculation with Arthrobacter ureafaciens Liulou 1

Method

Capacity of Arthrobacter ureafaciens liulou 1 to remediate polluted soil was assessed in a pot experiment performed in the manner described in Example 9. The pots used in the experiment had no holes to prevent the loss of atrazine and its metabolites with water flow. The pots contained soil (300 g dry weight equivalents) with 1.75 mg technical grade (≧97%) atrazine (5833 ng/g dry soil). Ten dry wheat seeds inoculated with (6.4±0.2)×10⁴ CFU of Arthrobacter ureafaciens liulou 1 per seed were sown to each pot. Non-inoculated seeds were sown to the control pots (Control W). Pots kept in the freezer (minus 22° C.) from the beginning of the experiment and pots without plants incubated together with the planted pots were additional controls (designated Control 0 and Control 1 respectively). The wheat plants were grown under sunlight with natural photoperiod of Jinan in September to October, 20-26° C. day temperatures and 15-20° C. at night. The soil was harvested 28 days post seeding. Each soil sample was the total volume of soil in a pot from which plant roots were accurately removed. The samples were freeze-dried, homogenized and kept at minus 22° C. prior analysis.

Contents of atrazine and its metabolites were analyzed in soils from 4 replicate pots. Atrazine and its metabolites were extracted from the soil samples by vortexing method.

For extraction, 1 g samples of freeze-dried homogenized soil were weighed into 2 mL tubes and mixed with 1 mL methanol-water solution (4:1 v/v). The tubes were secured horizontally in a MO BIO Vortex Adapter assembled on Vortex-Genie® 2 Vortex (MO BIO Laboratories, Inc., USA), vortexed at maximal speed for 1 h and leaved in the horizontal position for 16 h. After the incubation, tubes were vortexed for 5 min. The suspension was centrifuged at 8000 g for 3 min., and the supernatant was transferred to a 15 mL tube. Then 4 additional extractions were performed consisting of 30 min. vortexing and centrifugation at 8000 g for 3 min. All vortexing steps and the incubation after the first vortexing were performed at 30° C. The supernatants were combined and analyzed by HPLC-MS/MS in the manner described in Example 13. Parameters of MS/MS and limits of quantification for the analytes in soil are specified in Table 6. Recovery of the analytes from fortified soil samples was 90.5-101.2% for atrazine, 73.7-78.5% for 2-hydroxyatrazine, 89.6-104.7% for desethyl atrazine, 80.0-103.6% for desisopropyl atrazine, 91.4-104.1% for desethyl desisopropyl atrazine.

Results

HPLC-MS/MS analysis detected 5911.4±518.0 μg atrazine kg-1 soil from non-incubated Control 0 (Table 7), indicating that the extraction recovery was 101.3±8.9%. 2-hydroxyatrazine, desethyl atrazine, desisopropyl atrazine and desethyl desisopropyl atrazine were detected in the Control 0 soil as minor contaminants. Atrazine content in soil from the pots with bacterized plants was 125.1±8.4 μg kg-1, indicating that inoculation of wheat seeds with Arthrobacter ureafaciens strain liuloul resulted in degradation of nearly 97.9% of the herbicide applied.

Degradation of atrazine in non-planted (Control 1) and planted (Control W) soils amounted to 42-46% (Table 7). The analysis of atrazine metabolites in the non-treated soils from planted (Control W) and non-planted (Control 1) pots revealed accumulation of desethyl atrazine as a major contaminant and 2-hydroxyatrazine, desisopropyl atrazine, and desethyl desisopropyl atrazine as minor ones (Table 7). Application of Arthrobacter ureafaciens strain liuloul by means of seed inoculation completely prevented the accumulation of desethyl atrazine and desisopropyl atrazine in soil, and reduced desethyl atrazine concentration by 3.7 times compared to its initial level in the contaminated soil (Control 0). The content of desethyl desisopropyl atrazine in the treated soil was reduced by more than 4 times and the content of 2-hydroxyatrazine was reduced by nearly 25-33% compared to the non-planted Control 1 and planted Control W.

This example demonstrates that Arthrobacter ureafaciens strain liuloul applied to soil by means of seed inoculation before sowing (according to claims 8 and 10) remediates soil contaminated with atrazine by degrading atrazine and preventing accumulation or reducing the content of its metabolites 2-hydroxyatrazine, desethyl atrazine, desisopropyl atrazine and desethyl desisopropyl atrazine in soil.

TABLE 7
Soil remediation with Arthrobacter ureafaciens liulou 1

| | Analyte contents in control and bacterized soils, ng/g dry soil | | | |
|---|---|---|---|---|
| | Control 0 | Control 1 | | A. ureafaciens liulou 1 |
| | (Atrazine dose | (Atrazine dose | | (Atrazine dose 1.75 |
| | 1.75 mg/pot | 1.75 mg/pot | Control W | mg/pot (5833 ng/g |
| | (5833 ng/g dry | (5833 ng/g dry | (Atrazine dose 1.75 | dry soil), wheat, |
| | soil), no plants, | soil), no plants, | mg/pot (5833 ng/g | seed inoculation |
| | no inoculation, | no inoculation, | dry soil), wheat, no | with A. ureafaciens |
| Analyte | no incubation) | incubated) | inoculation, incubated) | liulou 1, incubated) |
| Atrazine | 5911.4 ± 518.0 | 3192.7 ± 387.5 | 3404.7 ± 272.7 | 125.1 ± 8.4 |
| Atrazine-2- | 67.7 ± 2.8 | 198.3 ± 15.0 | 226.1 ± 44.4 | 151.7 ± 8.6 |
| hydroxy | | | | |
| Atrazine- | 63.2 ± 1.5 | 1029.2 ± 114.6 | 975.4 ± 60.5 | 17.2 ± 2.3 |
| desethyl | | | | |
| Atrazine- | 20.6 ± 0.9 | 219.0 ± 18.5 | 234.3 ± 11.6 | 17.6 ± 1.6 |
| desisopropyl | | | | |
| Atrazine- | 0.90 ± 0.03 | 186.8 ± 15.6 | 178.1 ± 6.9 | 41.3 ± 1.8 |
| desethyl- | | | | |
| desisopropyl | | | | |

The invention claimed is:

1. An atrazine-degrading bacterium having all identifying characteristics of the strain Arthrobacter ureafaciens liulou 1 deposited in the China General Microbiological Culture Collection Center (CGMCC), under the No. 9667, on Sep. 10, 2014.

2. The bacterium of claim 1, wherein said bacterium completely degrades atrazine in a solution with a concentration of 25 mg/L in a stationary state within 1 week.

3. The bacterium of claim 1, wherein said bacterium is motile and actively spread on soft agar surfaces.

4. The bacterium of claim 1, wherein said bacterium applied to plant seeds at an effective amount from 10² to 10⁸ colony forming units per seed colonizes plant roots and produces populations sufficient to degrade soil atrazine.

5. The bacterium of claim 1, wherein said bacterium is capable of mobilizing insoluble phosphate.

6. The bacterium of claim 1, wherein said bacterium applied to the seeds of plants at an amount no less than 10² colony forming units per seed promotes the growth of seedling roots.

7. A method of remediation of liquids and soils contaminated with atrazine or related s-triazine compounds and for plant protection from atrazine or related s-triazine compounds, wherein the related s-triazine compounds are desethyl atrazine, desisopropyl atrazine, simazine, cyanazine, propazine, terbuthylazine, prometryn, ametryn, terbutryn, simetone, the method comprising applying a biological agent comprising an effective amount of a biologically pure culture of the bacterium of claim 1.

8. The method of claim 7, for remediation of soils contaminated with atrazine or related s-triazine compounds, the method comprising:
a) inoculating plant seeds with the biological agent comprising an effective amount of a biologically pure culture of the bacterium; and
b) sowing the inoculated seeds to contaminated soil and growing the plants.

9. The method of claim 7, for plant protection from atrazine or related s-triazine herbicides, comprising inoculating plant seeds with the biological agent comprising an effective amount of a biologically pure culture of the bacterium.

10. The method of claim 8, wherein the inoculated seeds can be stored, keeping an effective amount of the bacterium of claim 1 for at least 3 months from the date of the inoculation.

11. The method of claim 9, wherein the inoculated seeds can be stored, keeping an effective amount of the bacterium of claim 1 for at least 3 months from the date of the inoculation.


SEQUENCE LISTING (1 sequence)

SEQ ID NO: 1 - DNA, 1255 bp, Arthrobacter ureafaciens
tagtggcgaacgggtgagtaacacgtgagtaacctgcccttgactctgggataagcctgggaaactgggtctaataccggatatgactcctcatcgcatggtggggggtggaaagctttttgtggttttggatggactcgcggcctatcagcttgttggtggggtaatggcctaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagtagggaagaagccctctttgggggtgacggtacttgcagaagaagcgccggctaactacgtgccagcagccgcggtaatacgtagggcgcaagcgttatccggaattattgggcgtaaagagctcgtaggcggtttgtcgcgtctgctgtgaaagaccggggctcaactccggttctgcagtgggtacgggcagactagagtgcagtaggggagactggaattcctggtgtagcggtgaaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatggaccggaaagacctggaaacaggtgccccgcttgcggccggtttacaggtggtgcatggttgtcgtcagctcgtgtcgtgagatgttgggttaagtcccgcaacgagcgcaaccctcgttctatgttgccagcggttcggccggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagca